(19) United States
(12) Patent Application Publication
(10) Pub. No.: US 2026/0098853 A1
(43) Pub. Date: Apr. 9, 2026

(54) SELF-SENSING ELECTROSTATIC ACTUATORS AND METHODS FOR MEASURING THE MECHANICS OF SOFT MATERIALS

(71) Applicant: PURDUE RESEARCH FOUNDATION, West Lafayette, IN (US)

(72) Inventors: Alex CHORTOS, West Lafayette, IN (US); Pranav Nandan Parigi, Bangalore (IN)

(21) Appl. No.: 19/346,505

(22) Filed: Sep. 30, 2025

Related U.S. Application Data
(60) Provisional application No. 63/701,352, filed on Sep. 30, 2024.

(51) Int. Cl. G01N 33/483; G01N 3/40

(52) U.S. Cl. CPC G01N 33/4833; G01N 3/40

(57) ABSTRACT

Devices and methods for measuring the mechanical properties of soft materials, including biological materials, are disclosed. Embodiments include devices and methods wherein sensors and actuators are collocated with one another reducing complexities and uncertainties of traditional devices with separate sensors and actuators that are merely coupled to one another. Additional embodiments include devices and methods capable of applying large strains during the stimulation and sensing (deformation and measurement) process of determining the mechanical properties, which is some embodiments enables the determination of strain-dependent stiffness and viscoelasticity.

This application claims the benefit of U.S. Provisional Application No. 63/701,352, filed 30 Sep. 2024, the entirety of which is hereby incorporated herein by reference.

### FIELD

Embodiments of the present disclosure relate generally to systems and methods that measure the mechanical properties of soft materials, and to systems and methods that contact a soft material, including biological material, and measure the mechanical properties of the soft material.

### BACKGROUND

The mechanics of soft materials, such as biological tissues, can be important to measure. For example, the mechanics of biological tissues can be an indicator of disease in the biological tissue. While some indentation measuring methods (e.g., atomic force microscopy (AFM) and microscale indentation) may be usable with biological tissues, it was realized by the inventors of this present disclosure that in addition to requiring specialized equipment, these indentation methods can require operation in open air (potentially contaminating tissue samples, including engineered tissue samples) and/or are not suitable for wearable devices worn by a user. Some imaging approaches such as magnetic resonance elastography and ultrasound elastography being developed may also be capable of use with biological samples, the inventors of the present disclosure realized that these methods are generally very expensive and require the storage and computational analysis of extremely large amounts of data. And, while piezoelectric and magnetic devices capable of achieving high operation frequencies and low hysteresis may also be usable with biological samples, the inventors of the present disclosure realized that while systems combining these types of devices with sensors to measure the mechanical properties of skin (e.g., for longitudinal measurement of skin mechanics) may be possible, these types of systems typically include expensive and/or toxic materials and include stiff components that can influence the behavior of soft materials.

The inventors of the present disclosure therefore realized that problems exist with measuring the mechanics of biological samples and that improvements in measurement methods and apparatuses are needed.

Certain preferred features of the present disclosure address these and other needs and provide other important advantages.

### SUMMARY

Embodiments of the present disclosure provide an improved Self-Sensing Electrostatic Actuators and Methods for Measuring the Mechanics of Soft Materials.

In embodiments, the soft materials being measured exhibit a large elastic deformation, for example, materials with moduli less than 10 MPa.

Embodiments measure materials with moduli that are comparable to (which includes slightly greater than) or softer than the actuator, for example, some embodiments include an actuator with a modulus of approximately 1 MPa that measure tissues with a modulus of less than 2 MPa.

Measuring device embodiments disclosed herein have advantages in applications such as in situ monitoring and measuring of the mechanical properties of engineered tissues during growth. Further embodiments have advantages in being wearable devices for measuring skin mechanics that may be worn by a user while the measurements are being made.

Embodiments of the present disclosure are useful in biomedical applications (e.g., where measuring tissue stiffness is useful), such as in surgical instruments, bioreactors (e.g., for producing engineered tissues), and/or in drug testing devices.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims, but should not be construed to be a limitation unless expressly recited in a particular claim. Each embodiment described herein does not necessarily address every object described herein, and each embodiment does not necessarily include each feature described. Other forms, embodiments, objects, advantages, benefits, features, and aspects of the present disclosure will become apparent to one of skill in the art from the detailed description and drawings contained herein. Moreover, the various apparatuses and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

Some of the figures shown herein may include dimensions or may have been created from scaled drawings. However, such dimensions, or the relative scaling within a figure, are by way of example, and not to be construed as limiting.

FIG. 1A is a depiction of an actuator according to at least one embodiment of the present disclosure.

FIG. 1B is graphical depiction of a large amplitude voltage signal being used to actuate the actuator in FIG. 1A while a small-amplitude, high-frequency signal is used to measure the capacitance of the actuator in FIG. 1A.

FIG. 1C is a depiction showing how actuation of an actuator according to at least one embodiment of the present disclosure in contact with a second material will vary depending on the stiffness of the second material.

FIG. 1D is depiction of how the capacitance as a function of voltage can indicate the mechanical properties of the material that is in contact with the actuator according to at least one embodiment of the present disclosure.

FIG. 2A is a depiction of cantilevered actuators according to embodiments of the present disclosure behaving differently when free, bonded to an elastomer, and blocked.

FIG. 2B is a graph demonstrating the differences in variation of capacitance with respect to voltage for a free actuator, an actuator bonded to a soft elastomer, an actuator bonded to a stiff elastomer, and a blocked actuator according to one or more embodiments of the present disclosure.

FIG. 3 is a depiction of a suspended membrane actuator according to at least one embodiment of the present disclosure.

FIG. 4 is a depiction of cantilevered actuator according to at least one embodiment of the present disclosure.

FIG. 5 is a perspective and plan view of a conical actuator according to at least one embodiment of the present disclosure.

FIG. 6 is a depiction of a fiber actuator according to at least one embodiment of the present disclosure.

### DETAILED DESCRIPTION OF THE ILLUSTRATED EMBODIMENTS

For the purposes of promoting an understanding of the principles of the disclosure, reference will now be made to one or more embodiments, which may or may not be illustrated in the drawings, and specific language will be used to describe the same. It will nevertheless be understood that no limitation of the scope of the disclosure is thereby intended; any alterations and further modifications of the described or illustrated embodiments, and any further applications of the principles of the disclosure as illustrated herein are contemplated as would normally occur to one skilled in the art to which the disclosure relates. At least one embodiment of the disclosure is shown in great detail, although it will be apparent to those skilled in the relevant art that some features or some combinations of features may not be shown for the sake of clarity.

Any reference to “invention” that may occur within this document is a reference to an embodiment of a family of inventions, with no single embodiment including features that are necessarily included in all embodiments, unless otherwise stated. Furthermore, although there may be references to benefits or advantages provided by some embodiments, other embodiments may not include those same benefits or advantages, or may include different benefits or advantages. Any benefits or advantages described herein are not to be construed as limiting to any of the claims.

Likewise, there may be discussion with regards to “objects” associated with some embodiments of the present invention, it is understood that yet other embodiments may not be associated with those same objects, or may include yet different objects. Any advantages, objects, or similar words used herein are not to be construed as limiting to any of the claims. The usage of words indicating preference, such as “preferably,” refers to features and aspects that are present in at least one embodiment, but which are optional for some embodiments.

Specific quantities (spatial dimensions, temperatures, pressures, times, force, resistance, current, voltage, concentrations, wavelengths, frequencies, heat transfer coefficients, dimensionless parameters, etc.) may be used explicitly or implicitly herein, such specific quantities are presented as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated.

Measuring the mechanical properties of a material involves applying a force and measuring the resulting strain. This is often done using different devices. For example, a mechanical motor can apply a force while a strain gauge can measure the strain.

Embodiments of the present disclosure include an actuator 100 (e.g., a soft actuator with less than five (5) megapascals modulus (<5 MPa modulus)) that is activated by electrostatic forces as both the actuating component and the sensing component. Such an actuator 100 may be referred to as a dielectric elastomer actuator (DEA). These embodiments conceptually include a capacitor in which all components (e.g., the dielectric 110 and the electrodes 120) are made of soft materials, e.g., elastic materials with low (<5 MPa) modulus.

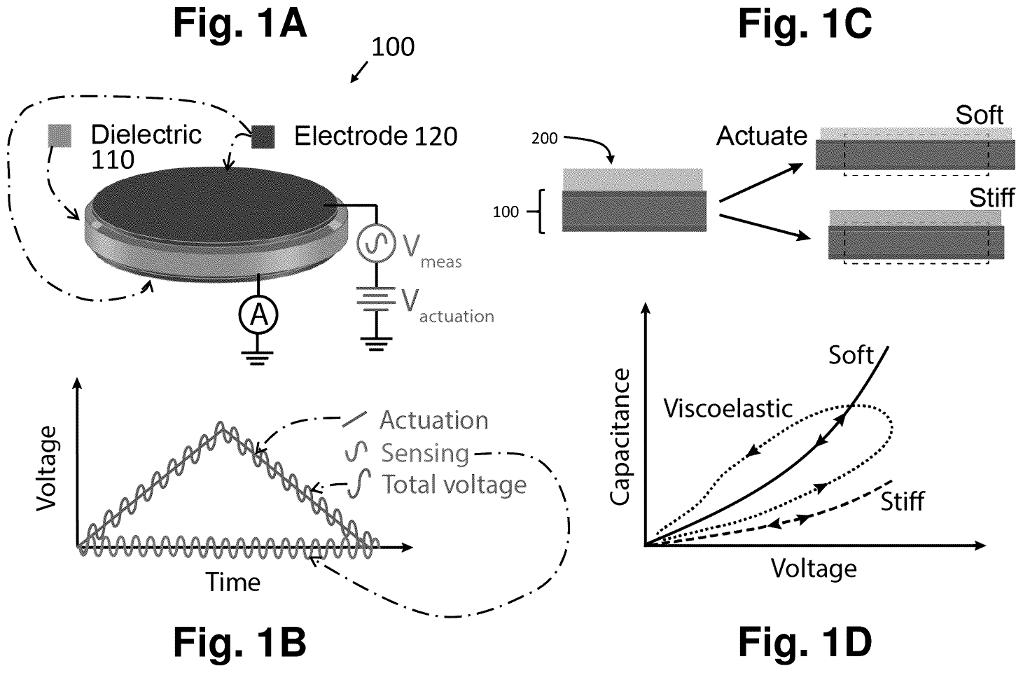

Referring to FIG. 1A, applying a voltage across a dielectric 110 (the dielectric 110 comprising a first material), such as by applying a voltage to opposing electrodes 120 (the electrodes comprising a second material) causes the dielectric 110 to actuate, e.g., compress in the vertical direction—the direction perpendicular to the planar surfaces of the electrodes 120. This compression in the vertical direction results in elongation in the horizontal direction—the direction parallel to the planar surfaces of electrodes 120—which is used for material sensing in at least some embodiments. Measuring the capacitance of the actuator 100 can provide information about the actuation state of the actuator 100. This ability to act as both an actuator and a sensor is referred to herein as self-sensing.

Referring to FIG. 1B, the self-sensing can be accomplished by applying a voltage (the “Total voltage” in FIG. 1B) that is a combination of a high-amplitude low-frequency voltage (the linear “Actuation” voltage in FIG. 1B) to actuate actuator 100 and a high-frequency low-amplitude sensing signal (the sinusoidal “Sensing” voltage following the x-axis in FIG. 1B) to measure the capacitance of the device.

Referring to FIG. 1C, when actuator 100 is connected to a third material (e.g., a sample 200, which may be a biological tissue sample) in such a manner that the actuator 100 cannot move independently of (e.g., cannot slide in relation to) the sample 200, the sample 200 constrains the deformation of the actuator 100. As further shown in FIG. 1C, the deformation of actuator 100 will be less when actuator 100 is connected to and restrained by a stiffer sample 200 than when actuator 100 is connected to and restrained by a softer sample 200. The dashed lines in FIG. 1C represent the original dimensions of the actuator. In the top representation for the actuated actuator 100 (which is connected to a soft sample 200) is horizontally wider and vertically thinner than the bottom representation for the actuated actuator 100 (which is connected to a stiff sample 200).

The relative difference between the modulus of the actuator 100 and the modulus of the sample 200 tend to affect the resolution of the sensor. It is also believed that there is no restriction on the relative difference between the modulus of actuator 100 and modulus of the sample 200. Nevertheless, while the range of stiffnesses capable of being sensed by sensors utilizing embodiments of the present disclosure is still being examined, it is expected that embodiments where the stiffness of an actuator 100 selected to measure the mechanics of a sample 200 is between one-tenth (0.1×) the stiffness of the sample 200 and one hundred times (100×) the stiffness of the sample 200 will be effective for measuring the mechanics of the sample 200.

The magnitude of the change in capacitance of actuator 100 as a function of voltage can be used to measure the mechanical properties (e.g., softness and/or stiffness) of the sample 200 with which the actuator is in contact. As shown in, FIG. 1D, the curves for softer materials 200 will appear above the curves for stiffer materials 200.

The modulus (e.g., stiffness) of the sample is typically measured at very low frequency, e.g., less than 10 Hz. However, the viscoelasticity of a tissue sample is related to the amount of energy dissipated by the sample as the sample deforms, which is measured at higher frequencies. Therefore, the ability of actuator 100 to operate at high frequencies (e.g., frequencies on the order of 10 Hz to 1,000 Hz, noting that the sensing signal can be at least 10 times higher) enables actuator 100 to measure additional properties of the sample, such as the viscoelasticity of biological tissue, and these high frequency embodiments can produce curves that exhibit some degree of hysteresis as shown in FIG. 1D.

Nevertheless, some embodiments utilize ionic actuators 100 that are unable to operate at high frequencies, and are therefore able to measure stiffness of a tissue sample but not the viscoelasticity of the tissue sample.

Still further embodiments utilize a liquid (e.g., a gel or oil) dielectric, and such actuators may be referred to as a hydraulically amplified self-healing electrostatic (HASEL) actuators. These embodiments are capable of applying different ranges of force, and may be useful in providing measurements of stiffer tissues. At least one example embodiment includes a liquid or gel filled pouch with electrodes on its surface. When a voltage is applied, the liquid or gel is displaced and causes actuation. Although these embodiments can apply forces differently from DEA actuators, these HASEL actuators show the same (or at least similar) capacitance variation with deformation and may be similarly used to measure stiffness as a sensor. While DEA actuators may have a modulus of <5 MPa, HASEL actuators can have a much higher stiffness (e.g., >100 MPa) and the liquid dielectric having a large dielectric constant.

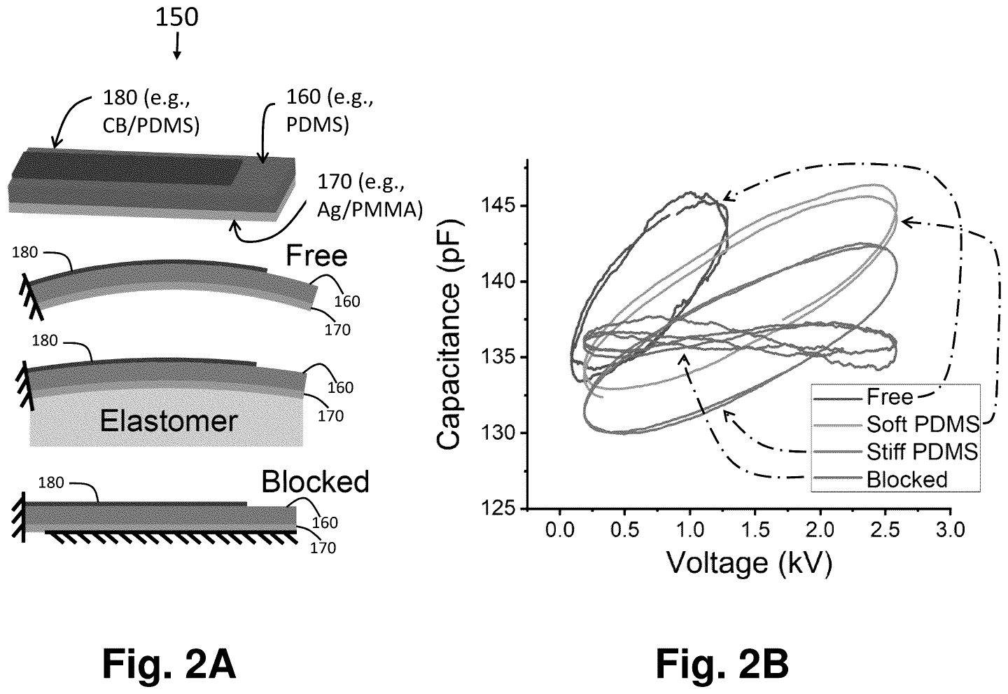

FIGS. 2A and 2B depict an actuator 150 according to embodiments of the present disclosure. Actuator 150 includes a first electrode 170 comprising a flexible (e.g., a material that is not so brittle that it will crumble when bent) yet inextensible (e.g., a modulus approximately two orders of magnitude greater than either the dielectric's modulus or the tissue's modulus, such as silver flakes suspended in polymethyl methacrylate (PMMA)) material, a dielectric 160 comprising a soft dielectric material (e.g., polydimethylsiloxane (PDMS)), and a second electrode 180 comprising a soft yet electrically conductive material (e.g., carbon black suspended in PDMS). In testing conducted on the actuator 150, actuator 150 was cantilevered and tested using four conditions to determine the properties of actuator 150:
free displacement (the cantilevered actuator 150 not being in contact with anything as shown in the second depiction from the top in FIG. 2A);
connected to a soft elastomer (as depicted in the second from the bottom depiction in FIG. 2A with the “Elastomer” being soft, e.g., a polydimethylsiloxane (PDMS) with a ratio of 1:20 with the crosslinker);
connected to a stiff elastomer (as depicted in the second from the bottom depiction in FIG. 2A with the “Elastomer” being stiff, e.g., a polydimethylsiloxane (PDMS) with a ratio of 1:10 with the crosslinker); and
blocked (the cantilevered actuator 150 being completely prevented from moving, such as by being placed on a glass slide, as shown in the bottom depiction in FIG. 2A).

As shown in FIG. 2B, in the blocked condition-D there was no change in capacitance in relation to the voltage since actuator 150 was not allowed to deform. In the free displacement condition-A there was a large change in capacitance in relation to voltage. In the soft elastomer contact condition-B there was an intermediate change in capacitance in relation to voltage that was less than the change in capacitance for the free displacement condition-A. And, in the stiff elastomer contact condition-C the magnitude of capacitance change in relation to voltage was between magnitude for the soft elastomer condition-B and the blocked condition-A. As such, actuator 150 was able to measure the nature of its boundary conditions, e.g., measure the nature of the material with which actuator 150 was in contact.

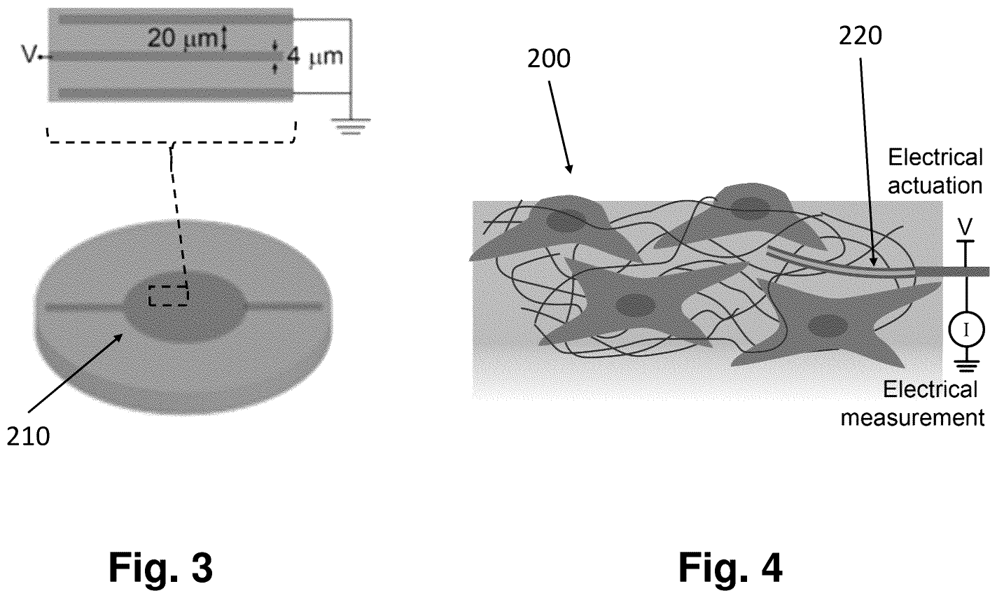

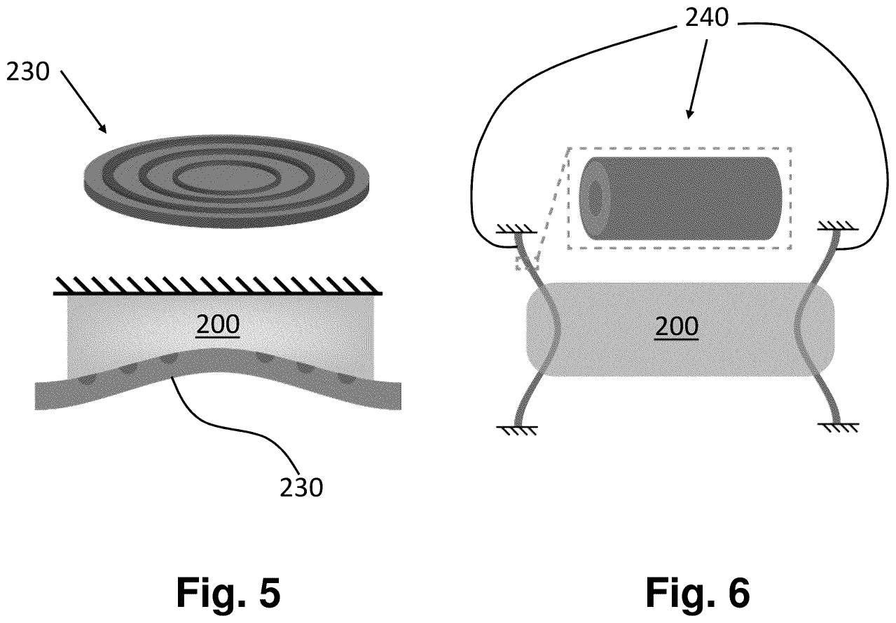

Other actuator embodiments include actuators of different geometries, such as suspended membrane actuator 210 (see, e.g., FIG. 3), cantilevered actuator 220 (see, e.g., FIG. 4 with the cantilever actuator 220 embedded in tissue and able to measure the local tissue modulus), conical actuator 230 (see, e.g., FIG. 5, which can be used for indentation measurements), fiber actuator 240 (see, e.g., FIG. 6, which can be used for measuring changes in tension in a biological sample), and fiber-based actuators where the dielectric and electrode layers are comprised of fibers that are bound together.

Additional embodiments of the present disclosure include utilizing actuators as disclosed herein (e.g., actuator 100 and/or actuator 150) during tissue engineering. For example, actuators 100/150 may be used to monitor the growth of tissues and provide feedback to the tissue engineers, such as incorporating actuators 100/150 as sensors into bioreactors that produced tissues to enable improved control over the tissue growth process and increase the quality of the tissues being grown.

Still further embodiments include utilizing actuators as disclosed herein (e.g., actuator 100 and/or actuator 150) during drug testing. For example, actuators 100/150 may be used in drug testing systems (e.g., advanced microphysiological systems) to measure and provide accurate feedback about the mechanics of cell cultures during drug testing, such as in vitro drug testing.

Yet further embodiments include utilizing actuators as disclosed herein (e.g., actuator 100 and/or actuator 150) during surgery, such as robotic surgery. For example, actuators 100/150 may be incorporated into surgical tools to enhance the surgeon's understanding of the tissue being manipulated.

Still further embodiments of the present disclosure include devices and methods for measuring the mechanical properties of soft materials wherein the sensors and actuators are collocated. Sensing mechanical deformation can be achieved by an actuator applying a deformation to a material and a sensor to measuring the deformation. Traditional systems have separate devices that perform the deformation and measurement functions (e.g., an actuator and a sensor that are coupled together). In contrast, embodiments of the present disclosure include an actuator that can sense its deformation state by measuring its own capacitance, so a single device performs both deformation and measuring functions. This reduces the complexity of the system and reduces uncertainties that can arise from coupling between an actuator and a sensor in traditional devices.

Yet additional embodiments of the present disclosure are capable of applying large strains during the stimulation/sensing (deformation/measurement) process. Many previous approaches have used technologies such as piezoelectric or ultrasound device to apply mechanical deformation, but these approaches apply small amplitudes of strain, which can measure the Young's modulus of materials. However, most biological materials have nonlinear mechanical properties and therefore have tangent moduli that change as the strain increases. The ability of embodiments of this disclosure to apply large strains allow the measurement of strain-dependent stiffness and viscoelasticity

Advantages of embodiments of the present disclosure include:
actuators with a large range of strains (>10%) and frequencies (up to 1000 Hz) that allow measurement of strain-dependent and frequency-dependent mechanical properties;
low cost actuators since some embodiments can be made of low cost materials such as polymers and carbon-based conductors;
mechanical compliance with, for example, biological applications;
actuators that require no external components other than electrical connections to the two electrodes, which is in stark contrast to, for example, imaging approaches that require large equipment to be located at specific locations; and
actuators that are compatible with miniaturization and/or multiplexing.

Reference systems that may be used herein can refer generally to various directions (e.g., upper, lower, forward and rearward), which are merely offered to assist the reader in understanding the various embodiments of the disclosure and are not to be interpreted as limiting.

To clarify the use of and to hereby provide notice to the public, the phrases “at least one of A, B, . . . and N” or “at least one of A, B, . . . . N, or combinations thereof” or “A, B, . . . and/or N” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. As one example, “A, B and/or C” indicates that all of the following are contemplated: “A alone,” “B alone,” “C alone,” “A and B together,” “A and C together,” “B and C together,” and “A, B and C together.” If the order of the items matters, then the term “and/or” combines items that can be taken separately or together in any order. For example, “A, B and/or C” indicates that all of the following are contemplated: “A alone,” “B alone,” “C alone,” “A and B together,” “B and A together,” “A and C together,” “C and A together,” “B and C together,” “C and B together,” “A, B and C together,” “A, C and B together,” “B, A and C together,” “B, C and A together,” “C, A and B together,” and “C, B and A together.”

While examples, one or more representative embodiments and specific forms of the disclosure have been illustrated and described in detail in the drawings and foregoing description, the same is to be considered as illustrative and not restrictive or limiting. The description of particular features in one embodiment does not imply that those particular features are necessarily limited to that one embodiment. Some or all of the features of one embodiment can be used or applied in combination with some or all of the features of other embodiments unless otherwise indicated. One or more exemplary embodiments have been shown and described, and all changes and modifications that come within the spirit of the disclosure are desired to be protected.

### ELEMENT NUMBERING

Table 1 includes element numbers and at least one word used to describe the element and/or feature represented by the element number. However, none of the embodiments disclosed herein are limited to these descriptions. Other words may be used in the description or claims to describe a similar member and/or feature, and these element numbers can be described by other words that would be understood by a person of ordinary skill reading and reviewing this disclosure in its entirety.

TABLE 1

| | |
|---|---|
| 100 | actuator |
| 110 | dielectric |
| 120 | electrode |
| 150 | actuator |
| 160 | dielectric |
| 170 | first electrode |
| 180 | second electrode |
| 200 | sample |
| 210 | suspended membrane actuator |
| 220 | cantilevered actuator |
| 230 | conical actuator |
| 240 | fiber actuator |

What is claimed is:

1. An actuator for determining the mechanical properties of a sample tissue, comprising:
a dielectric;
a first electrode coupled to a first side of the dielectric;
a second electrode coupled to the second side of the dielectric;
wherein the first electrode is
flexible,
inextensible in comparison to the dielectric, and
inextensible in comparison to the sample tissue.

2. The actuator of claim 1, wherein
the dielectric includes polydimethylsiloxane (PDMS); and
the first electrode includes silver flakes suspended in polymethyl methacrylate (PMMA) and the modulus of the first electrode is two orders of magnitude greater than either the modulus of the dielectric or the modulus of the sample tissue; and
the second electrode includes carbon black suspended in polydimethylsiloxane (PDMS).

3. The actuator of claim 1, wherein the modulus of the first electrode is two orders of magnitude greater than either the modulus of the dielectric or the modulus of the sample tissue.

4. The actuator of claim 3, wherein the first electrode includes silver flakes suspended in polymethyl methacrylate (PMMA).

5. The actuator of claim 1, wherein the dielectric includes polydimethylsiloxane (PDMS).

6. The actuator of claim 1, wherein the second electrode includes carbon black suspended in polydimethylsiloxane (PDMS).
